(19) United States
(12) Patent Application Publication
(10) Pub. No.: US 2005/0038322 A1
(43) Pub. Date: Feb. 17, 2005

(54) Imaging endoscope

(75) Inventor: Michael S. Banik, Bolton, MA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: SCIMED Life Systems

(21) Appl. No.: 10/639,040

(22) Filed: Aug. 11, 2003

(51) Int. Cl. A61B 1/00

(52) U.S. Cl. 600/129; 600/182; 600/160

(57) ABSTRACT

An endoscopic imaging system includes an endoscope with a beam deflecting mechanism at or adjacent its distal end for directing a beam of illumination light over an area of interest. Reflected light is gathered by one or more lenses and supplied to a light sensor and an image processor/computer that produces an image of the tissue. In one embodiment, the beam deflecting mechanism comprises a pair of mirrors that are oscillated such that light is scanned in a raster pattern over the area of interest.

### FIELD OF THE INVENTION

The present invention relates to medical devices in general, and in particular to imaging endoscopes.

### BACKGROUND OF THE INVENTION

One of the most common methods for non-invasively screening an internal body cavity of a patient is with an imaging endoscope. Such endoscopes are elongated devices that are inserted into the body cavity. Light is delivered through an illumination channel of the endoscope and reflected light is gathered by one or more lenses that are coupled to an imaging channel. Light from the imaging channel is transmitted out of the endoscope and supplied to a camera or other viewing device so that a physician can examine the internal body tissue. Typical cameras connected to the endoscope typically include a solid state image sensor such as a CCD array.

One problem with conventional imaging endoscopes is their relatively low resolution. For example, on a 3.5 mm square CCD array, the resolution is limited to approximately 850K pixels. Another problem is their high cost. At a current price of approximately $350 each, the cost of such image sensors alone makes it impractical to design single use or disposable endoscopes. Instead, such endoscopes need to be sterilizable so they can be used on many different patients. In order to withstand the high temperature and/or harsh chemical environment used in sterilization, conventional endoscopes are made to be relatively stiff and rugged. However, the same factors that contribute to the long life of an endoscope also reduce its ability to be inserted into some body cavities. Therefore, there is a need for an endoscope that has a higher resolution and can be manufactured at a cost that makes it practical to be a single use item.

### SUMMARY OF THE INVENTION

To address these and other concerns, the present invention is an imaging endoscope having a light beam directing mechanism for steering a beam of illumination light over an area of interest. Light reflected from tissue in the area of interest is received by a photo sensor that converts the light into a corresponding electrical signal. Electrical signals are combined in an image processor to produce an image of the tissue.

In one embodiment of the invention, the light deflecting mechanism comprises a pair of mirrors that are moved by oscillating microelectrical machines (MEMS) that steer the light in a raster fashion over the area of interest. In one embodiment, light is directed to the moving mirrors via an input optical fiber that extends from a proximal end to a distal end of the endoscope.

Images of tissue can be stored in a database and analyzed by a computer to determine the likelihood that an image contains a particular type of tissue such as a cancerous lesion. If a lesion is detected, the intensity of the illumination light may be selectively increased to ablate the tissue in situ. A display is provided to show a physician or other user the image of the tissue.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 illustrates a disposable imaging endoscope and image detection system in accordance with one embodiment of the present invention;

FIG. 2 shows one embodiment of a light deflection mechanism at the distal end of the endoscope in accordance with another embodiment of the present invention; and

FIG. 3 shows yet another embodiment of a light deflection mechanism in accordance with the present invention.

### DETAILED DESCRIPTION OF THE PREFERRED EMBODIMENT

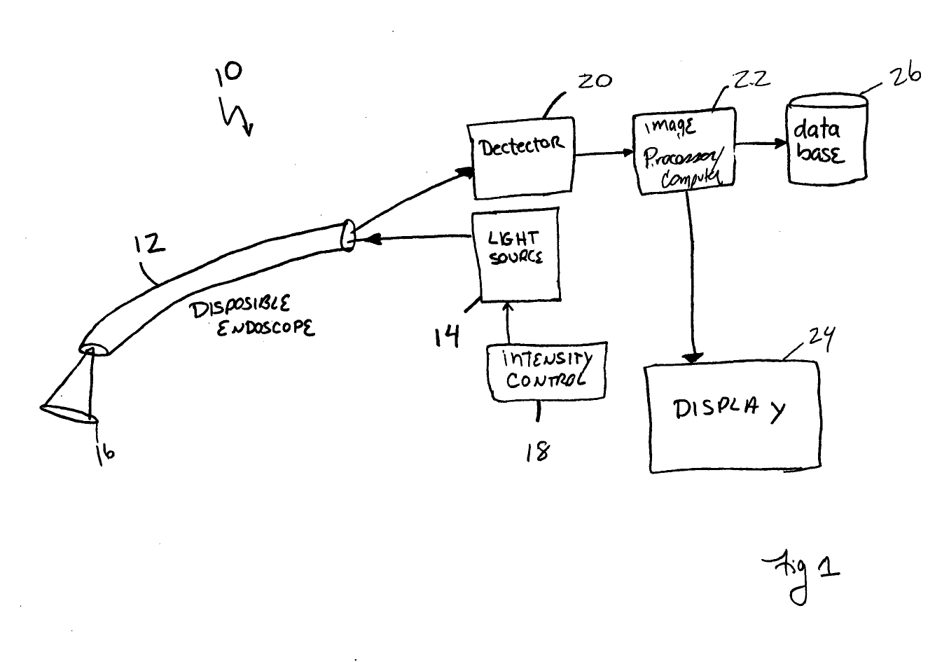

FIG. 1 illustrates one embodiment of a disposable imaging system 10 in accordance with the present invention. The imaging system 10 includes a disposable endoscope 12 generally comprising an elongate tube that directs light from a light source 14 onto an area of interest 16 that is within an internal body cavity (not shown). Light reflected from the area of interest 16 is gathered and returned through the endoscope 12 to a photo detector 20. The photo detector 20 generates electronic signals that are proportional to the intensity of the received light. The electronic signals produced by the photo detector 20 are supplied to an image processor/computer 22 that combines the electronic signals produced over the area of interest and creates an image of the tissue. Images produced by the image processor/computer 22 are displayed on a display device 24 such that a physician or other user can view the internal body tissue of a patient. The images from the image processor may be recorded and stored in a database 26 for recall by the image processor/computer 22. In addition, the endoscope 12 may include one or more lumens for the passage of surgical instruments in order for a physician to obtain a biopsy or perform other procedures in the body cavity of the patient.

One of the benefits of the endoscope 12 is that because it lacks a camera chip within the endoscope itself, it can be made for a sufficiently low cost such that it can be considered a single use or disposable item. Therefore, the costs associated with sterilizing the endoscope are not incurred for the user. Furthermore, the endoscope 12 can be made more flexible than conventional endoscopes because it does not need to withstand the high temperatures or other harsh chemical environments typically required for sterilizable endoscopes.

If the physician sees a tissue sample that appears cancerous or should otherwise be removed from the patient, the physician adjusts an intensity control 18 of the light source 14. By adjusting the light source to a sufficient intensity, any suspicious tissue can be ablated in situ. For example, if the light source 14 comprises a laser, the power of the laser can be selectively increased or decreased by the intensity control 18 to ablate the tissue or collect images.

As an aid to diagnosing tissue samples viewed by the physician, the image processor/computer 22 can analyze images of the tissue to determine if they represent cancerous or other particular tissue types. Such analysis by the processor/computer types can be based on the pathology of known lesions. Dyes or other markers of specific tissue types can be detected by the image processor/computer and used to identify the tissue type. Alternatively, an image can be measured by the image processor/computer 22 according to a number of criteria such as the length, roundness, eccentricity, texture or other morphological features known by image cytometrists to highlight or identify particular tissue types. New images of a tissue sample can be compared against these criteria and potentially suspicious tissue can be highlighted on the display 24 for a physician to view prior to determining whether the tissue should be ablated or removed surgically.

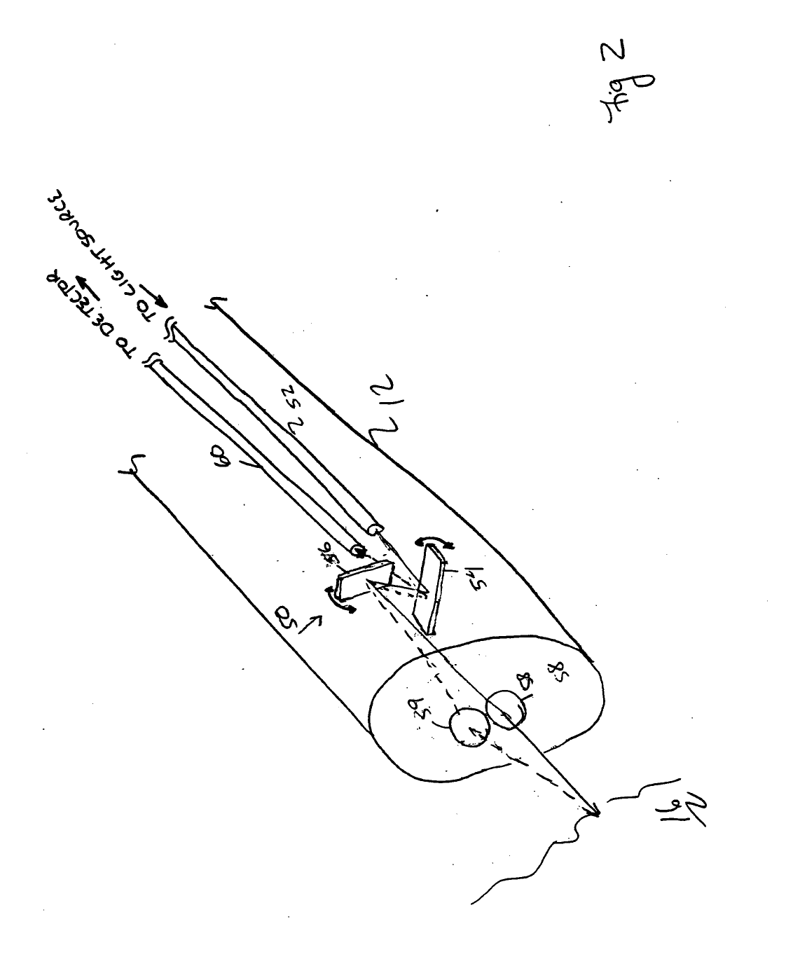

FIG. 2 shows a portion of one embodiment of an endoscope 12 including a beam deflecting mechanism disposed at the distal end. Light is delivered to the distal end of the endoscope by a first optical fiber 52. Light from the optical fiber 52 is directed to a pair of oscillating mirrors 54, 56. The first mirror 54 is used to direct the light beam back and forth along a scan line. Light from the first mirror 54 is reflected onto the second mirror 56 which is moved back and forth to move the position of the scan lines over an area of interest. Together the mirrors 54, 56 operate to direct the illumination light from the fiber 52 in a raster scan pattern. Light reflected off the second mirror 56 is passed through one or more lenses 58 to focus the light on the tissue in the area of interest 16. Light reflected or emitted from the tissue in the area of interest 16 is passed through the one or more lenses 59 to the mirrors 56, 54 where it is directed to a return optical fiber 60. The optical fiber 60 is coupled to the photo detector 20 as shown in FIG. 1.

The beam deflection mechanism 50 is preferably made of one or more microelectronic machines (MEMS) that are inexpensive enough to manufacture such that the endoscope 12 can be considered a single use or disposable item. Details of one suitable mechanism for driving the mirrors 54, 56 are fully described in U.S. Pat. Nos. 6,245,590 and 6,331,909, assigned to Microvision, Inc. of Bothell, Wash. and herein incorporated by reference. However, it will be appreciated that there are other mechanisms for moving the mirrors, including electric motors, piezoelectric crystals or other devices that can move the mirrors to move the illumination light over an area with a repeating pattern that may be other than a raster pattern.

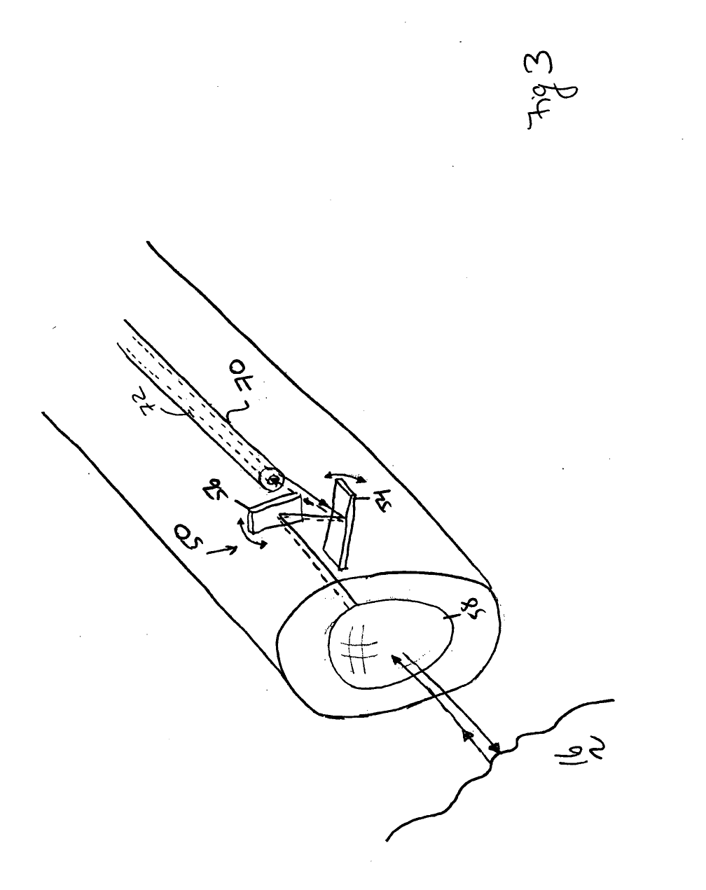

FIG. 3 shows an alternative embodiment of an endoscope in accordance with the present invention. The beam deflection mechanism 50 includes a pair of oscillating mirrors 54, 56 as described above. However, light is delivered to the beam deflection mechanism by a multicore fiber 70. The fiber includes an outer cladding 72 in which light is delivered to the beam deflecting mechanism. After being deflected by the mirrors 54, 56, the light passes through a set of one or more lenses 61 that focus the light on the tissue. Light reflected from or generated by the tissue is passed through the set of one or more lenses 61 where it is directed back onto the mirrors 54, 56 and into a central core of the multicore fiber 70. Because the input and output optical fibers are part of the same multicore fiber and thus are axially aligned, a single set of one or more lenses 61 can be used at the distal end of the endoscope. The central core of the multicore fiber 70 is connected to the photo detector 20 as shown in FIG. 1, while the outer cladding 72 is connected to the light source 14 as shown in FIG. 1.

The photo detector 20 as shown in FIG. 1 comprises a photodiode or other light sensor that produces an electronic signal that is proportional to the intensity of the light it receives. The intensities detected over an entire scan area are supplied to the image processor/computer 22 in order to produce a final image of the tissue. The details by which the image of the tissue is created are not considered important to the understanding of the present invention and are generally known to those of ordinary skill in the art of computer aided image construction and optics.

While the preferred embodiment of the invention has been illustrated and described, it will be appreciated that various changes can be made therein without departing from the spirit and scope of the invention. For example, it is possible to incorporate a solid state light source or other light emitting device near the distal end of the endoscope and to eliminate the need for an input optical fiber to transfer light from an external light source to the beam deflection mechanism. Similarly, the light sensor could be located within the endoscope itself to eliminate the optical fiber that carries reflected light out of the endoscope. One or more lenses would direct reflected light into the sensor and wires would carry the corresponding electrical signals to a remote image processor/computer. Therefore, the scope of the invention is to be determined from the following claims and equivalents thereof.

1. An endoscopic imaging system, comprising:
a source of illumination light;
an endoscope having an input optical fiber;
a beam deflection mechanism for directing illumination light received on the input optical fiber over an area of interest, the beam deflection mechanism including at least one mirror that directs the illumination light over a scan line and at least one other mirror that moves the scan line over the area of interest;
an output optical fiber;
one or more lenses that gather reflected light and direct it to the output optical fiber;
a detector that receives light from the output optical fiber;
an image processor/computer coupled to the detector for producing an image of the area of interest; and
a display for displaying the image.

2. The endoscopic imaging system of claim 1, wherein the input and output optical fibers are different fibers.

3. The endoscopic imaging system of claim 1, wherein the input and output optical fibers are part of a multicore fiber.

4. The endoscopic imaging system of claim 1, wherein the mirror that directs light back and forth over a scan line and the mirror that moves the scan line over the area of interest are moved by microelectronic machines (MEMS).

5. The endoscopic imaging system of claim 1, wherein the mirror that directs light back and forth over a scan line and the mirror that moves the scan line over the area of interest are moved by piezoelectric crystals.

6. The endoscopic imaging system of claim 1, further comprising a light source with a selectable power output that can be increased to ablate tissue in situ.

7. A disposable imaging endoscope, comprising:
a catheter having a proximal end and a distal end;
at least one optical fiber within the catheter connectable to a light source;
an oscillating beam deflection mechanism adjacent the distal end of the catheter for directing light from the light source over an area of interest;
one or more lenses at the distal end of the catheter for collecting light reflected from the area of interest; and
at least one optical fiber for carrying the collected light to an optical detector.

8. The disposable imaging endoscope of claim 7, wherein the optical fiber that directs light from the light source and the optical fiber that carries light to the optical detector are part of a multicore fiber.

9. The disposable imaging endoscope of claim 7, wherein the oscillating beam deflection mechanism comprises at least two mirrors that direct light over a raster pattern.

10. The disposable imaging endoscope of claim 9, wherein the two mirrors are moved by MEMS devices.

11. The disposable imaging endoscope of claim 9, wherein the two mirrors are moved by piezoelectric crystals.

12. A disposable imaging endoscope, comprising:
a catheter having a proximate end and a distal end;
means for supplying a beam of illumination light;
an oscillating beam deflection mechanism for directing the illumination light over a regular, repeating pattern;
one or more lenses for gathering reflected light; and
means for delivering the reflected light to a light sensor.

13. The disposable imaging endoscope of claim 12, wherein the means for delivering a beam of illumination light comprises one or more optical fibers coupled to an external light source.

14. The disposable imaging endoscope of claim 12, wherein the means for delivering a beam of illumination light comprises a light source within the catheter.

15. The disposable imaging endoscope of claim 12, wherein the means for delivering the light to a light sensor comprises one or more optical fibers.

16. The disposable imaging endoscope of claim 12, wherein the means for delivering the light to a light sensor comprises one or more lenses that direct light into a light sensor that is within the catheter.
